(19) United States
(12) Patent Application Publication
(10) Pub. No.: US 2006/0173482 A1
(43) Pub. Date: Aug. 3, 2006

(54) Device for insertion of lacrimal stents

(76) Inventor: Jeremy S Melker, Gainesville, FL (US)

Correspondence Address: Timothy H Van Dyke;Beusse Brownlee Wolter Mora & Maire, Suite 2500, 390 North Orange Avenue, Orlando, FL, 32801, US

(21) Appl. No.: 10/549,421

(22) PCT Filed: Apr. 28, 2004
(86) PCT No.: PCT/US04/13233

§ 371 Date: Sep. 15, 2005

Related U.S. Application Data
(60) Provisional application No. 60/466,031, filed on Apr. 28, 2003.

(51) Int. Cl. A61M 29/00

(52) U.S. Cl. 606/191

(57) ABSTRACT

Disclosed herein is system for inserting lacrimal stents utilizing a rigid tapered probe and a flexible, blunt-tipped probe, wherein both probes provide a channel for directing a guidewire. The guidewire assists in efficiently positioning lacrimal stents, while minimizing possible injury to the lacrimal apparatus structures.

### FIELD OF THE INVENTION

The present invention relates to the field of lacrimal stents, devices for manipulation of such stents, and novel methods in the area of inserting small stents.

### BACKGROUND OF THE INVENTION

Lacrimal fluid or tears are continuously supplied from the lacrimal gland located laterally and superiorly of the eye through the upper lacrimal duct to the conjunctival sac in which the eyeball is partially encased. Thus, the lacrimal fluid washes across the sclera and other conjunctival components and also the cornea.

The lacrimal apparatus consists of a series of ducts which allow the natural drainage of lubricating tears from the eyes into the nasal cavity. Upper and lower lid punctae, located near the medial aspect of the lids, lead to lacrimal canaliculi. These, in turn, drain to the lacrimal sac, which then becomes confluent with the nasolacrimal duct, the ultimate pathway by which tears reach the nasal cavity.

Quite often a permanent closure occurs in the canaliculi, the lacrimal sac, or the nasolacrimal duct, whereupon the lacrimal fluid no longer can be disposed of in the normal maimer resulting in chronic tearing (epiphora). Such closure or stenosis can result from congenital anomalies, following surgical manipulation of the eye or surrounding structures, accident, inflammation or other disease, or advancing age. In addition, the canaliculi may become scarred from conjunctival infection. Epiphora likewise can result from blockage of the canaliculi by a cilium or by streptothrix concretions. In severe cases permanent stenosis occurs and a dacryocystorhinostoiny is indicated.

Upon occurrence of blockage in the lacrimal drainage ducts, the eye continuously brims over with tears much to the discomfort, annoyance, and embarrassment of the individual so affected. A much more serious consequence is the potential for the stagnating tears to result in infection and inflammatory irritation of the mucous membrane with proliferation of the epithelium, hyperemia, and a purulent exudation into the conjunctiva.

Lacrimal duct stenting is commonly performed as a portion of surgical procedures in which the lacrimal apparatus is violated. The goal of stent placement is to prevent scaring of drainage pathways, thereby avoiding the complication of epiphora. Additionally, stents may be placed for congenital malformations of the lacrimal apparatus, or when the apparatus has been disrupted as the result of trauma.

Current techniques for duct cannulation and stenting rely on enlargement of the 10 punctae with tapered dialators, followed by the passage of silastic tubing attached to a blunt tipped probe or suture. See for example, U.S. Pat. No. 4,380,239 issued to Crawford and Wainman. The silastic tubing has a probe attached at both ends; one of which is used to cannulate the lower puncta, the other the upper puncta. The probes and attached tubing are passed through the punctae, canaliculi, lacrimal sac, and nasolacrimal duct remnant and into the nasal cavity. Finally, the probes are detached from the tubing, the ends of which are then tied together. The silastic tubing may then be left in place for weeks to months.

Initial passage of tapered dilators is typically not difficult. Their rigid design allows for precise control and finely tapered points can reliably be directed into even the smallest punctae. On the other hand, the successful cannulation of the punctae with the blunt probes (which are attached to the silastic tubing) can be quite difficult. By necessity, the shafts of the probes must be flexible to allow passage within the acutely angled lacrimal apparatus. Additionally, their tip must be blunt enough to prevent violation of the walls of the lacrimal apparatus with inadvertent injury to surrounding orbital structures. As a result of these design requirements, it is difficult for the surgeon to gain the mechanical advantage necessary to successfully caimulate the punctae. Often the probe will partially seat in the puncta, only to slip out of position whenever attempts at advancement are made. This can lead to frustration for the surgeon, significant operating delays, excessive lid edema, and place the cornea at increased risk of abrasion because of prolonged manipulation.

Older patents discuss ways of using probes to assist guiding the stent into place, but such techniques fall short of addressing the surgical problems and complications. For example, U.S. Pat. No. 4,305,395 discloses the use of a probe that is inserted into a closed ended stent. The probe increases the rigidity of the stent which assists initially with inserting the stent into the punctae. However, the lack of flexibility of the probe makes it difficult to navigate the angles of the lacrimal apparatus. Furthermore, the '395 patent could not incorporate larger sized stents since it does not teach a method of implementing a dilation instrument. Accordingly, there is a need for an improved system and method for inserting lacrimal stents.

### SUMMARY OF THE INVENTION

The subject invention is directed to novel system and methods for inserting lacrimal stents. The subject system faciliatates easy insertion of both small and large stents, while minimizing injury to the lacrimal apparatus and orbit.

One object of the invention is to provide a novel tapered probe to assist in the placement of a guidewire into the puncta and canaliculi of the patient in need. The novel tapered probe is preferably cannulated so that a guidewire can be passed through the cannula. Preferably still, the novel tapered probe comprises a cannula that passes from the tip of the tapered probe to a sideport on the tapered probe.

Another object of the invention pertains to a novel flexible, blunt-tipped probe that is cannulated. Preferably, the cannula passes from the tip of the flexible, blunt-tipped probe to a sideport defined thereon.

A further object of the invention pertains to a kit comprising a caimulated, rigid, tapered probe and a flexible, blunt-tipped probe. In addition, the kit may include silastic tubing for insertion and use as a lacrimal stent. The cannulated, rigid, tapered probe and flexible, blunt-tipped probe of the kit preferably comprise a channel defined within which span from the tip of the probes to a site on the side of the probes, sideport.

Yet another object of the subject invention is directed to a novel method for placement of a lacrimal stent that comprises insertion of a rigid tapered dilator probe in the puncta. Once a critical opening size is achieved, a second tapered probe comprising a central lumen leading to a sideport is placed in the puncta. A blunt-ended guidewire is passed through the sideport, through the lumen, and out the tip of the second tapered probe. The guidewire is guided safely into the puncta and directed into the canaliculi.

Once the guidewire is in place, the second tapered probe is removed. A blunt-tipped, flexible probe having a central lumen and sideport is obtained. The guidewire is positioned through the tip, through the lumen, and out the sideport of the flexible probe. Once the flexible probe is inserted into the canaliculi a desired amount, the guidewire is pulled out of the patient and flexible probe. The guidewire assists in guiding the flexible probe into the puncta by keeping the vector of applied force centered therein, and into the canaliculi, and can be swiftly and easily removed from the patient. Once the flexible probe is inserted into canaliculi, it can be advanced in the lacrimal apparatus in standard fashion.

The foregoing has outlined some of the more pertinent objectives of the present 15 invention. These objectives should be construed to be merely illustrative of some of the more prominent features and applications of the invention. Many other beneficial results can be attained by applying the disclosed invention in a different manner of modifying the invention as will be described.

It is to be understood that the foregoing general description and the following detailed description are exemplary and explanatory only and are not to be viewed as being restrictive of the present, as claimed. These and other objects, features and advantages of the present invention will become apparent after a review of the following detailed description of the disclosed embodiments and the appended claims.

### BRIEF DESCRIPTION OF THE DRAWINGS

FIG. 1 shows a side view of prior art blunt-tipped, flexible probes, FIG. IA, and prior art rigid tapered dilators, FIG. 1B-C.

FIG. 2 shows a side view of one embodiment of the subject invention. FIG. 2A shows a rigid tapered dilator comprising a channel defined from its tip to a location on its side. FIG. 2B shows a blunt-tipped flexible probe comprising a channel defined from its tip to a location on its side.

FIG. 3 shows a diagram of a preferred method embodiment of the subject invention.

FIG. 4 shows a view of the lacrimal apparatus, FIG. 4A, and shows a stent embodiment positioned in the upper and lower punctae FIG. 4B.

### DETAILED DESCRIPTION OF THE PREFERRED EMBODIMENTS

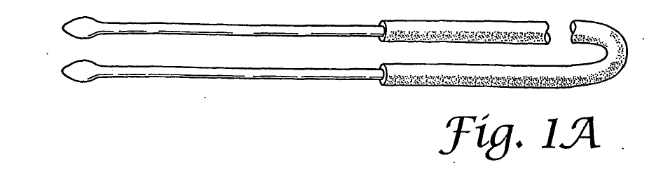

FIG. 1 shows examples of blunt-tipped flexible probes (FIG. 1A, commonly known as “Crawford Probes”) and rigid tapered dilators (FIGS. 1B and C) that are currently used for inserting lacrimal stents in patients with a need for such stents. The probes and dilators shown in FIG. 1 represent just a few examples of current devices that may be modified according to the teachings herein to achieve probes and dilators for use with the subject methods. See www.fci-opthalmics.com; U.S. Pat. Nos. 5,437,625; 5,318,513; and 6,083,188 for a non-exhaustive sampling of other probes, stents, and related devices that may be modified in accordance with the principles of the subject invention. For example, such devices may include a channel that spans from one end, preferably tip end, of the device to a side port, or through the opposite end, wherein a guidewire may pass through the channel to assist the physician in guiding a stent into the lacrimal apparatus, as taught herein.

FIG. 2 shows a dilator 204 and probe 202 comprising a channel 210, 211, respectively, that spans from the tip 212, 2t3, respectively, of the dilator 204 or probe 202 to a sideport 214, 215, respectively. Those skilled in the art, in view of the teachings herein, will appreciate that lacrimal insertion instruments, in addition to those shown in FIG. 1, may be modified to include a channel comprising the features of the channel illustrated in FIG. 2, i.e., extending from the tip of the instrument to a sideport. In an alternative embodiment, the dilator or analogous instrument may be fully cannulated from one end of the instrument to the other.

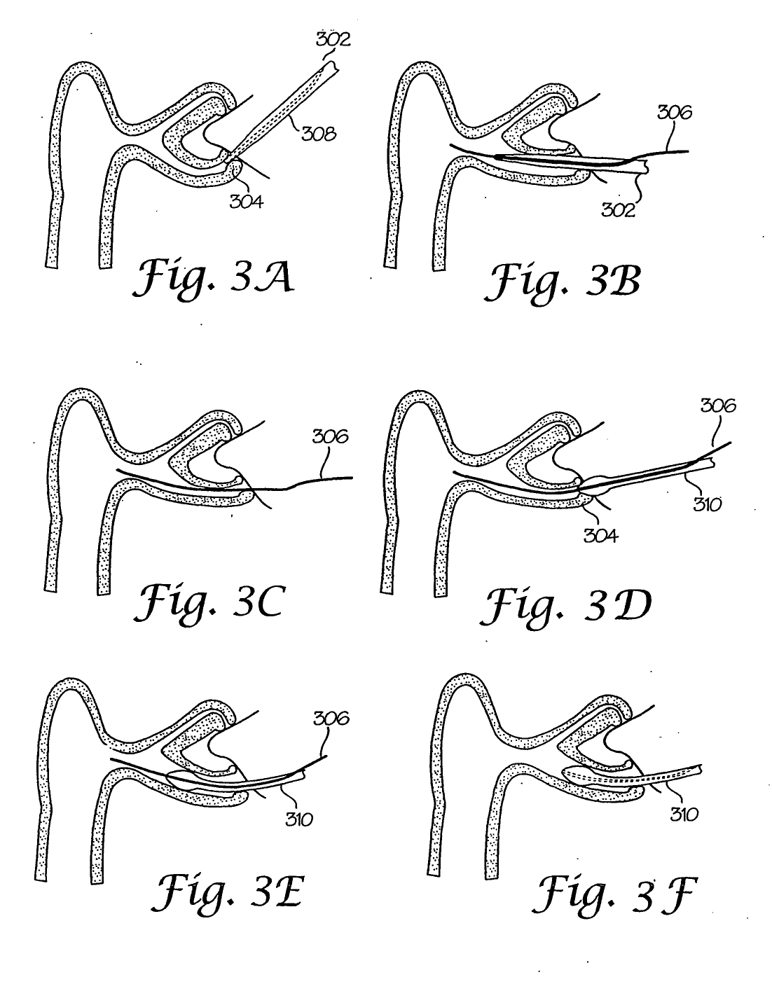

FIG. 3A-F represents a stepwise diagram of a preferred embodiment of the subject methods for inserting a lacrimal stent. FIG. 3 shows the insertion of a rigid tapered dilator 302, such as that shown in FIG. 2A, into the puncta 304 of a patient. When the rigid tapered dilator 302 has been inserted at the appropriate depth for desired dilation of the puncta 304, a guidewire 306 is passed through the channel 308 of the dilator 302. The guidewire 306 is inserted to the appropriate depth; and the dilator 302 is then removed from the canaliculi 303 and puneta 304, whereby the guidewire 306 remains in place. A blunt-tipped, flexible probe 310, such as that shown in FIG. 2B, is passed over the guidcwire 306 and into the puncta 304. The guidewire 306 assists the physician in guiding the probe 310 into the puncta 304. When the probe 310 is inserted at a desired depth, the guidewire 306 is passed out of the probe 310. The probe 310 is then advanced through eanaliculi and appropriate duct(s) in conventional fashion.

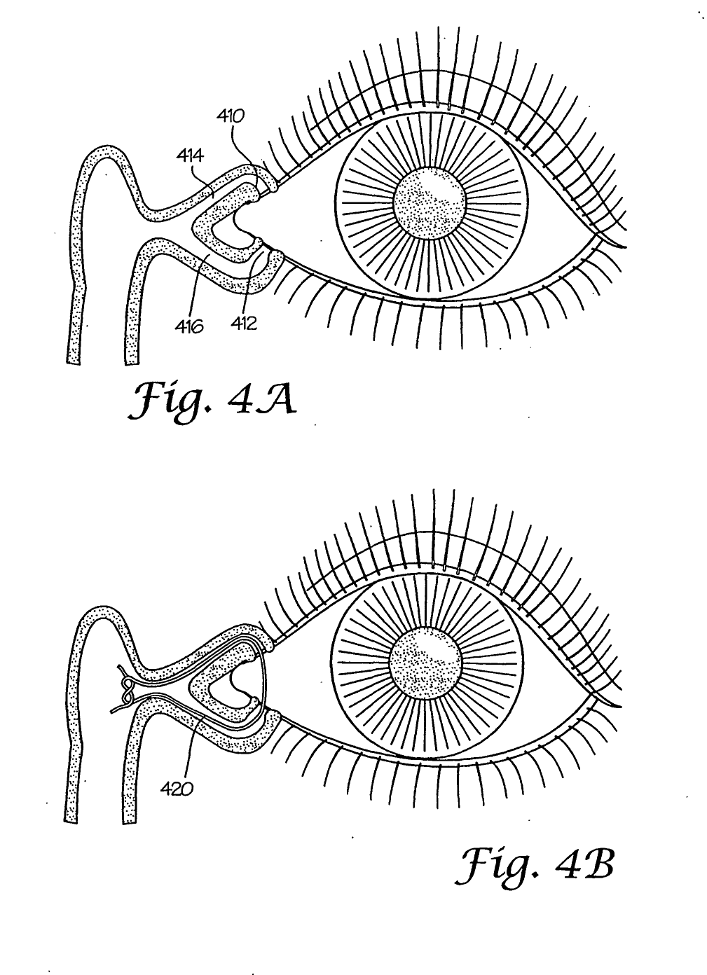

FIG. 4A illustrates the anatomy of a portion of the lacrimal apparatus including the upper 410 and lower 412 punctae, upper 414 and lower 416 canaliculi, and the lacrimal sac 418. FIG. 4B illustrates the stent 420 in place after the ends have been inserted through the upper and lower punctae, probes cut, and ends tied together in the nasal cavity.

All patents, patent applications, publications, texts and references discussed or cited herein are incorporated by reference to the same extent as if each individual publication or patent application was specifically and individually set forth in its entirety. Nothing herein is to be construed as an admission that the invention is not entitled to antedate such disclosures by virtue of prior invention. In addition, all terms not specifically defined are first taken to have the meaning given through usage in this disclosure, and if no such meaning is inferable, their normal meaning. Where a limitation is described but not given a specific term, a term corresponding to such limitation may be taken from any references, patents, applications, and other documents cited herein, or, for an application claiming priority to this application, additionally from an Invention Disclosure Statement, Examiner's Summary of Cited References, or a paper otherwise entered into the file history of this application.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims. Thus, for the above variations and in other regards, it should be understood that the examples and embodiments described herein are for illustrative purposes only and that various modifications or changes in light thereof will be suggested to persons skilled in the art and are to be included within the spirit and purview of this application and the scope of the appended claims.

1. A probe useful in inserting a lacrimal stent comprising a proximal end and a distal end, said distal end being tapered to facilitate entry into a puncta of a patient, wherein said probe comprises a channel extending from said distal end to a sideport on said probe positioned between said proximal end and said distal end.

2. The probe of claim 1, wherein said distal end is rigid.

3. A probe useful in inserting a lacrimal stent comprising a proximal end and a distal end, said distal end being flexible and comprising a blunt tip, wherein said probe comprises a channel extending from said distal end to a sideport on said probe positioned between said proximal end and said distal end.

4. The probe of claim 3, wherein said proximal end has affixed thereto a lacrimal stent tube.

5. A kit comprising at least one first probe comprising a proximal end and a distal end, said distal end channel being tapered to facilitate entry into a puncta of a patient, wherein said at least one first probe comprises a channel extending from said distal end to a sideport on said probe positioned between said proximal end and said distal end; and at least one second probe comprising a proximal end and a distal end, said distal end being flexible and comprising a blunt tip, wherein said probe comprises a channel extending from said distal end to a sideport on said probe positioned between said proximal end and said distal end; whereby said first and second probes are useful in assisting with insertion of lacrimal stents in a patient in need thereof

6. The kit of claim 5 wherein said at least one first probe comprises a rigid, tapered probe.

7. A method for inserting a lacrimal stent comprising inserting a tapered probe into a puncta of a patient to dilate said punta to a desired opening size, said tapered probe comprising a rigid distal end, a proximal end, and a channel extending from said distal end to a sideport between said distal end and said proximal end of said tapered probe; passaging a guidewire comprising a distal end and a proximal end through said tapered probe such that said distal end of said guidewire is passaged through said sideport, out said distal end of said tapered probe, into said puncta, and positioned in the patient's lacrimal apparatus at a desired depth; and removing said tapered probe.

8. The method of claim 7 further comprising passaging said guidewire through a second probe comprising a distal end, a proximal end, and a channel extending from said distal end to a sideport between said distal end and said proximal end of said second probe such that said guidewire is passaged through said distal end, through said channel and out said sideport; and inserting said second probe into said puncta, wherein said second probe is guided into said patient's lacrimal apparatus at a desired depth; and removing said guidewire from said patient.

9. The method of claim 8, wherein said distal end of said second probe is flexible and comprises a blunt tip.

10. The method of claim 8, wherein said second probe comprises a silastic tubing affixed to its proximal end.

11. The method of claim 10, wherein said silastic tubing comprises a first end and a second end; and wherein said first end is affixed to said second probe and said second end is affixed to a third probe comprising a distal end, a proximal end, and a channel extending from said distal end to a sidcport between said distal end of said third probe.

12. The method of claim 11, wherein said patient comprises an upper puncta and a lower puncta and wherein said second probe is inserted into said lacrimal apparatus through said lower puncta and said third probe is inserted into said lacrimal apparatus through said upper puncta.
